(19) United States
(12) Patent Application Publication
(10) Pub. No.: US 2019/0083154 A1
(43) Pub. Date: Mar. 21, 2019

(54) SURGICAL INSTRUMENT

(71) Applicants: JRI ORTHOPAEDICS LIMITED, Sheffield South Yorkshire (GB); SURGICAL INNOVATIONS LIMITED, Leeds Yorkshire (GB)

(72) Inventors: Kristopher CARVER, Washington Tyne and Wear (GB); Edward Richard Cornell DRAPER, Twickenham Greater London (GB); Ian John FLATTERS, Sheffield South Yorkshire (GB); Giles Francis Mansfield PROFFITT, Sheffield South Yorkshire (GB)

(73) Assignees: JRI ORTHOPAEDICS LIMITED, Sheffield South Yorkshire (GB); SURGICAL INNOVATIONS LIMITED, Leeds Yorkshire (GB)

(21) Appl. No.: 16/085,266

(22) PCT Filed: Mar. 16, 2017
(86) PCT No.: PCT/GB2017/050729

§ 371 Date: Sep. 14, 2018

(30) Foreign Application Priority Data
Mar. 18, 2016 (GB) 1604659.1

(51) Int. Cl. A61B 17/88; A61B 17/16

(52) U.S. Cl. CPC A61B 17/8805; A61B 17/1604; A61B 17/1617; A61B 2017/00314; A61B 17/1637; A61B 17/1664; A61B 17/1624

(57) ABSTRACT

A surgical instrument for minimally-invasive arthroscopic use in a constrained joint space, including a handle assembly, a shaft assembly including a shaft having a longitudinal shaft axis and a lumen therethrough and connected to handle assembly at proximal end of shaft and an operative portion including a working tip with a tool mounted thereon at distal end of shaft. Operative portion is selectively moveable between a first configuration substantially aligned with longitudinal shaft axis, and a second, deployed, configuration in which at least part of operative portion has a selectable working direction defined by the angle between tool and longitudinal shaft axis and a selectable lateral displacement defined by the linear distance between working tip and longitudinal shaft axis, measured at a central location point at which tool is mounted and perpendicular to longitudinal shaft axis, the working direction and lateral displacement being selectable independently of one another.

The present invention relates to a surgical instrument for minimally invasive arthroscopic use in a constrained joint space. Particularly, but not exclusively, the invention relates to a surgical instrument for removing the osteoarthritic lesion and for delivering a regenerative medicine product (RMP) to a target region.

### BACKGROUND

Osteoarthritis (OA) is a condition characterised by the degeneration of articular cartilage and underlying bone. OA causes pain and stiffness in the affected joint or joints, which can make it difficult to move the affected joints. OA commonly occurs in the hip, knee, and small joints of the hands. Treatment for osteoarthritis includes lifestyle changes, medication, supportive treatments, surgery and complementary or alternative therapy.

WO2010148125 A1 discloses a microfracture method and device for articular cartilage repair. At least two discrete channels, spaced a predetermined distance apart from one another are created through the cartilage and underlying subchondral bone, down into the marrow cavity of the bone. Blood and marrow elements, including mesenchymal stem cells, growth factors, and other healing factors and proteins, are released from the subchondral marrow space into the defect through the perforations to form a “super clot.” The “super clot” provides an enriched environment for the formation of new chondral tissue in the defect. After channel formation, higher levels of negative pressure may be applied to draw out marrow components, stem cells, and blood from the marrow cavity into the chondral defect to accelerate formation of a super clot. Whilst microfracture has been shown to provide successful short-term results, techniques with longer-term results that restore, rather than repair cartilage are preferable.

In earlier stages of OA, lesions are typically small and grow outwards as the disease progresses. Whilst bone is a good healing reserve due to the presence of mesenchymal stem cells in the bone marrow, cartilage has a comparatively reduced healing capability. Consequently, damage occurring whilst removing an osteoarthritic lesion must be minimised. Studies have shown that the quality of the cutting edge when osteoarthritic lesions are removed is important for cell survival at the wound site of the remaining cartilage. When blunt cutters, such as a curette are used, there is a notable increase in cell death around the proximity of the cut edge, as compared to cartilage cut with a sharp scalpel. Increased precision and reduced surface roughness promote cell survival at the wound site. Cutting techniques known in the art for cutting cartilage include lasers, drills, burrs, radiofrequency ablation, thermal ablation and curettes. Curettes are relatively blunt due to their curved shape and consequently put a region of stress on the cartilage in use. Indeed, none of the aforementioned known cutting techniques give a controlled edge or desired precision.

Other treatments for OA include hip replacement surgery or hip resurfacing surgeries. However, these treatments are generally reserved for end stage treatment of OA. These techniques are performed in open surgery procedures, which are highly invasive and require hip distraction. Addressing minor, early stage osteoarthritic lesions with open surgery is undesirable, because open surgery generally inflicts a lot of stress on the body.

Minimally invasive methods of performing surgery in joints of the human or animal body are becoming increasingly popular. Arthroscopy is typically used for joints having a straight line of access, such as the knee. In arthroscopic surgery, at least one incision is made and often, a corresponding arthroscopy portal may be placed into at least one incision. Various tools may then be interchangeably used throughout the procedure. In the knee, arthroscopic surgery can be performed in a direction (the working direction) that is the same as the direction in which the target region of the joint is accessed (the access direction). In the example shown in FIG. 1, an arthroscope 2 passes, via an incision, through an arthroscopy portal (not shown) through skin 4 and ligaments 6 of the knee 100, below the patella 8 to contact a target region 10 in the head 12 of the femur 14. A medical professional can then operate on the target region 10 without the arthroscope 2 being required to depart from the axis 16 of the direction of access; the axis 16 of the access direction is collinear with the axis of the working direction. The skilled person will appreciate that both the access and working direction can be easily made collinear for any target region located in a variety of locations on the articulating surfaces of the knee joints simply by moving the lower leg through the natural range of motion of the knee 100. For example, should the target region 10 be located elsewhere on the articulating surfaces of the knee joint, a medical professional can readily move the lower leg to move the tibia 18 and fibula 20 relative to the femur 14, so as to expose the target region of the femoral condyles 12 and tibial plateau 22, without having to re-engineer the arthroscope 2.

In contrast, arthroscopic procedures for constrained joint spaces, such as the hip and shoulder, are less established. A constrained joint space typically has complex geometry that means that the axes of the access direction and the working direction are not always collinear for every given target region. The target region is usually on an articulating surface. Some target regions may require an arthroscope to be able to work over or around a curve, or around a corner, due to anatomical obstructions, however in any joint, there is little space to work in between articulating surfaces. For example, the skilled person will appreciate that in the case of the hip joint, some locations on the articulating surface cannot be operated on with the axes of the access direction and working direction collinear. For some possible locations of target region, simply moving the upper leg of the patient through its natural range of motion will not sufficiently expose the target region. Indeed, ball and socket joint spaces usually fall within the meaning of “constrained joint space” as used herein.

Medical devices with articulating end portions are known, for example that disclosed in US2010280526. An articulating shaft portion is illustrated in FIGS. 5 and 6 thereof in which a slat assembly “provides the necessary support for articulation as well as for the other forces generated during cutting and/or manipulation” (paragraph [0049]). However, the example end effectors listed in paragraph [0054] and the only specific procedure described (meniscectomy at paragraph [0066]) do not indicate suitability for this device for the specific needs of arthroscopy in confined joint spaces.

Some known devices with articulating shaft portions are, by definition, flexible and therefore not able to reliably withstand the working load involved in arthroscopic procedures.

Access to the central part of the hip joint requires distraction of the joint. Hip distraction is invasive, puts stress on the body and causes the patient pain during post-operative recovery. Additionally, the use of hip distraction imposes a time limit on the surgery, meaning surgery might be rushed, or steps omitted. Arthroscopic surgery is occasionally used to treat femoral acetabular impingement (FAI), a condition wherein the head of the femur does not have full range of motion within the acetabulum. The condition also results in damage to the surrounding cartilaginous tissue. However, due to the time limit on the surgery, repair to damaged cartilage is usually prioritised last and is rushed or even omitted if there is not sufficient time remaining once joint impingement has been addressed.

There is a need for surgical instrumentation for treating an osteoarthritic joint surface in a minimally invasive manner, particularly for joints with complex geometrical constraints, such as the hip, shoulder or ankle.

### BRIEF SUMMARY OF THE DISCLOSURE

Aspects and embodiments of the invention relate to a surgical instrument, a cartridge for use as a reservoir of implantable material for use in a surgical instrument, a surgical apparatus, a minimally invasive arthroscopic method of preparing a target region of a constrained joint space and a minimally invasive arthroscopic method of delivering implantable material to a target region of a constrained joint space, as claimed in the appended claims.

In accordance with an aspect of the present invention there is provided a surgical instrument for minimally-invasive arthroscopic use in a constrained joint space, the surgical instrument comprising:

a handle assembly;

a shaft assembly including:

a shaft having a longitudinal shaft axis and a lumen therethrough and being connected to the handle assembly at a proximal end of the shaft; and

an operative portion including a working tip with a tool mounted thereon at a distal end of the shaft,

wherein, in use, the operative portion is selectively moveable between a first configuration in which the operative portion is substantially aligned with the longitudinal shaft axis, and a second, deployed, configuration in which at least part of the operative portion has:

a selectable working direction defined by the angle between the tool and the longitudinal shaft axis; and

a selectable lateral displacement defined by the linear distance between the working tip and the longitudinal shaft axis, measured at a central location point at which the tool is mounted thereon and perpendicular to the longitudinal shaft axis,

the working direction and lateral displacement being selectable independently of one another.

Independent selection of the working direction and the lateral displacement enables improved access for the surgeon to a constrained joint space. The instrument can advantageously be used to treat lesions within a constrained joint space, such as the hip joint.

In some embodiments, the operative portion includes an articulation segment which is said working tip or is intermediate said working tip and said shaft. The articulation segment may have actuation means capable of moving the operative portion between the first configuration and the deployed configuration, as desired.

The actuation means may comprise one or more rods, cables, pneumatic or hydraulic means located within the shaft lumen and actuable via said handle.

In an embodiment, the actuation means comprises an actuation rod and there may be a rod link at a distal end of and pivotable with respect to said actuation rod. Use of a rod is advantageous as its rigidity enables it to be both pushed and pulled (unlike a cable). A rod is also less likely to change its properties over time (a cable may become slacker through use, for example).

The actuation rod may have a radially extending formation or rod link end at or near a proximal end thereof, the formation being axially fixed with respect to a rod link end housing and axially moveable therewith so as to operate said articulating element.

The surgical instrument may further comprise a locator tube which is selectively axially translatable along the longitudinal shaft axis, the locator tube being capable of moving the rod link end housing axially. The locator tube may be selectively axially translatable along the longitudinal shaft axis by virtue of a screw thread at one end thereof.

Optionally, the locator tube screw thread engages a screw thread on a rotatable position selector, rotation of which position selector causes the axial translation of the locator tube.

Optionally, the surgical instrument further comprises locking means which, in said deployed configuration, lock the operative portion in the deployed configuration with respect to the shaft in order that a working load can be applied in the working direction. In an embodiment, the locking means comprises the selection of a sufficiently narrow screw thread pitch on the locator tube and/or position selector. The working load may be in the range 3N to 10N. Locking means advantageously enables the surgeon to retain the operative portion in a specifically selected position during a step of minimally invasive arthroscopic surgery, for as long as is required. The selected lateral displacement and working direction may also be retained even if the shaft assembly is removed from and replaced in the handle assembly.

The lateral displacement may be selectable by selecting an angle of articulation of the articulation segment with respect to the longitudinal shaft axis.

The working direction may be selectable by selecting the angle at which the tool is mounted on the working tip. The working direction may also be selectable by adjusting the angle at which the tool is mounted on the working tip.

In an embodiment, the shaft assembly is removable and replaceable via the proximal end of the handle assembly. This allows a user to switch between one or more different shaft assemblies during an arthroscopic procedure. A subsequent shaft assembly may have a same type of working tip at its operative portion as a previous shaft assembly, or it may have a different type of working tip at its operative portion. The ability to change working tip type by exchanging just the shaft assembly may advantageously reduce operating time and complexity.

The surgical instrument may additionally comprise locator means for recording positional information relating to the shaft assembly so that said shaft assembly or another shaft assembly can be replaced in a repeatable position after removal from the handle assembly. This advantageously reduces operating time, as the user will not have to spend time adjusting the positioning of a subsequent shaft assembly to position it with the same working direction and lateral displacement as a previous shaft assembly.

It may be that in use, the shaft, aligned with the access direction, has no direct line of sight to the target region.

In an embodiment, the locator means comprises the locator tube whose axial position remains unchanged while the shaft assembly is being removed and replaced.

In some embodiments, the tool is moveable in a closed loop path, for example a circle, with respect to a central location point fixed with respect to the shaft. This provides ample range of motion for the operative portion and enables a working tip to contact a large range of areas within a constrained joint space. It may be that said central location point is on an axis aligned with said working direction.

In an embodiment, said tool comprises a cutting element capable of producing a cut surface at said target region, the cut surface having one or more selectable characteristics, for example size, depth, orientation, shape and surface topography. This advantageously enables the surgeon to prepare a target region and to tailor the characteristics of the prepared target region. The cutting element may be a blade, a laser cutter, ultrasonic cutter or a fluid jet cutter. The cutting element may be moveable in a closed loop path with respect to the central location point fixed with respect to the shaft, at which the tool is mounted on the working tip so as to make a perimeter cut along said closed loop path at said target region. This advantageously provides a continuous perimeter cut, which is associated with lower cell mortality rates at the site of the cut. In some embodiments the cutting element is capable of cutting to remove material from an area within said perimeter cut.

In some embodiments, said tool comprises delivery means capable of delivering implantable material to a target region. This advantageously mitigates the need for a separate surgical instrument for delivering an implantable material. Optionally, the working tip includes a wiper tool moveable in said closed loop path to wipe excess implantable material from the target region. This ensures that implantable material is accurately placed. It may be that the wiper tool or an additional component of the working tip can compress the implantable material within the target region.

Optionally, the operative portion includes a curing tool for curing implantable material.

In some embodiments, said delivery means comprises a nozzle connectable to a reservoir containing said implantable material. This advantageously reduces the amount of storage space needed to store said implantable material and surgical instruments. Said reservoir may be located within said handle assembly or within the lumen of the shaft. Optionally, said reservoir is a removeable and replaceable cartridge. The handle assembly may include an outer housing having an aperture at a proximal end through which said reservoir can be installed, removed and replaced.

In some embodiments, said reservoir may contain a plunger capable of expelling implantable material from said reservoir, the plunger being actuable via said handle assembly. The nozzle may be in fluid connection with said reservoir via the lumen of the shaft.

According to another aspect of the invention there is provided a cartridge for use as a reservoir of implantable material suitable for use in a surgical instrument as described in any of the preceding paragraphs, the cartridge comprising a chamber containing implantable material and an outlet through which the implantable material can exit the chamber. The implantable material may be in the form of a powder, an injectable liquid, a morcellated solid, a gel, or any combination thereof.

The implantable material may comprise two components, stored in separate chambers until mixing thereof is desired, optionally further comprising a frangible membrane between the separate chambers. The cartridge may include a plunger for expelling said implantable material through said outlet.

The cartridge may include one or more end caps. In some embodiments, said outlet is connectable to said nozzle of the delivery means. Optionally, said chamber is refillable with implantable material.

According to another aspect of the invention there is provided surgical apparatus for minimally-invasive arthroscopy comprising:

a surgical instrument as described in any of the preceding paragraphs;

clamping means for clamping said handle assembly in a fixed position with respect to the target region so that said shaft assembly can be replaced in a repeatable position after removal from the handle assembly.

Optionally, said clamping means comprises an operating table clamp and/or an adjustable arm clamp. The surgical apparatus may comprise a cartridge as described in any of the paragraphs above.

According to another aspect of the invention there is provided a minimally-invasive arthroscopic method of preparing a target region of a constrained joint space using surgical apparatus as described in any of the paragraphs above comprising the steps of:

inserting a shaft assembly into said housing assembly;

moving said shaft assembly along an access direction towards the target region with the operative portion in its first configuration;

actuating said instrument to move the operative portion into its second, deployed configuration having a first working direction and a first lateral displacement;

clamping the housing assembly with respect to the target region; and

actuating said working tip in the first working direction to prepare the target region.

The method may further comprise the steps of:

actuating said instrument to move the operative portion back into its first configuration;

removing the shaft assembly from the instrument and replacing it with a second shaft assembly;

moving said second shaft assembly along the access direction towards the target region with the operative portion its first configuration;

actuating said instrument to move the operative portion into its second, deployed configuration in a second working direction and/or a second lateral displacement; and

actuating said working tip in the second working direction to prepare the target region.

Optionally, the first working direction and the second working direction are the same. Optionally, the first lateral displacement and the second lateral displacement are the same.

The method steps may be repeated for one or more additional target regions.

According to another aspect of the invention there is provided a minimally-invasive arthroscopic method of delivering implantable material to a target region of a constrained joint space using surgical apparatus as described in any of the paragraphs above comprising the steps of:

inserting a shaft assembly whose working tip comprises delivery means capable of delivering implantable material to said target region into said housing assembly;

moving said shaft assembly along an access direction towards the target region with the operative portion in its first configuration;

actuating said instrument to move the operative portion into its second, deployed configuration; and

actuating said delivery means to deliver implantable material to the target region.

The method may further comprise the steps of:

inserting a shaft assembly whose working tip comprises wiping means capable of wiping excess implantable material from said target region into said housing assembly;

moving said shaft assembly along an access direction towards the target region with the operative portion in its first configuration;

actuating said instrument to move the operative portion into its second, deployed configuration; and

actuating said wiping means to wipe excess implantable material from said target region.

The methods may additionally include the step of curing the implantable material delivered to the target region.

### BRIEF DESCRIPTION OF THE DRAWINGS

Embodiments of the invention are herein, by way of example only, with reference to the accompanying drawings, in which:

FIG. 1 (PRIOR ART) is a schematic representation of an arthroscopic procedure on a knee joint;

FIG. 2 is a schematic representation of a hip joint showing a surgical instrument approaching along an access direction;

FIG. 3 is a schematic representation of a hip joint showing two target regions, each having a working direction;

FIG. 4A is a schematic representation of a lesion requiring treatment;

FIG. 4B shows the lesion after preparation of its wall edge;

FIG. 4C shows the lesion after filling with a regenerative medicine product;

FIGS. 5 and 6 (PRIOR ART) are reproduced from US2010280526 and have been annotated to illustrate the working direction and lateral displacement of this prior art instrument. Reference numerals in these figures relate to features of US2010280526;

FIGS. 7A and 7B show the distal end of a surgical instrument in the first and the second, deployed, configurations respectively;

FIG. 7C is a side view of the surgical instrument of FIG. 7B, partly in section;

FIG. 8 indicates how the cutting element can make a perimeter cut by moving in a closed loop path indicated by the arrows;

FIGS. 9A and 9B show the distal end of a surgical instrument in the first and the second, deployed, configurations respectively, having a different working tip to that of FIGS. 7A and 7B;

FIGS. 10A-10D show the distal end of a surgical instrument in the deployed configuration, having a cutting tool which can be independently articulated;

FIG. 11 indicates how the cutting element can remove material from an area within the perimeter cut;

FIG. 12A is a side view of a surgical instrument in the second configuration in which the operative portion is orientated so as to be capable of operating on a target region in a working direction;

FIG. 12B indicates how the delivery means can deliver implantable material to a target region;

FIGS. 13A-13H show different embodiments of the surgical instrument, having different tools at the operative portion;

FIG. 14 is a cross-sectional view of an embodiment of the instrument with the distal end at the left of the Figure, the operative portion being in the first, undeployed position;

FIG. 15 shows the instrument of FIG. 14 in the second, deployed, position;

FIG. 16 shows the instrument of FIG. 14 with the shaft assembly temporarily removed;

FIG. 17 shows the instrument of FIG. 14 with the shaft assembly replaced with one having a different working tip and a different working direction;

FIG. 18 is a perspective view of another handle assembly;

FIG. 19A is a perspective view of another handle assembly;

FIG. 19B is a perspective view of a shaft assembly;

FIG. 20 is a side view of a handle assembly and shaft assembly assembled together and mounted on a table clamp;

FIG. 21 shows how the instrument is used to access a lesion site in the hip joint;

FIG. 22 shows a surgical table arm clamp with adjustable instrument support;

FIG. 23 shows a reservoir for implantable material, connectable to delivery means;

FIG. 24 shows a cartridge with end caps in place;

FIG. 25 shows the cartridge of FIG. 24 with the end caps removed;

FIG. 26 shows how a cartridge can be loaded into the surgical instrument; and

FIG. 27 shows how two cartridges can be loaded into the surgical instrument.

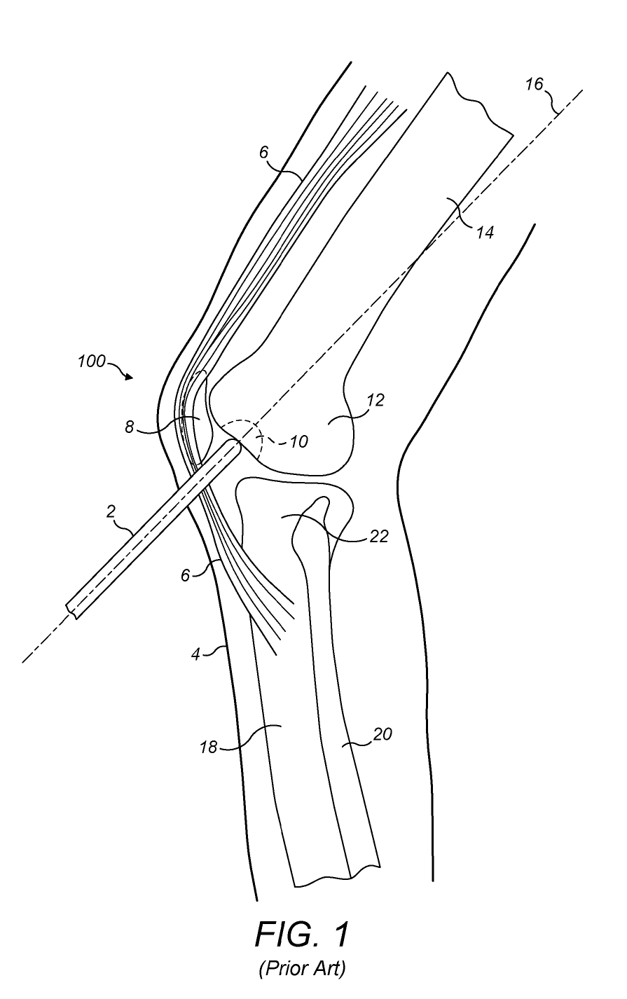

### DETAILED DESCRIPTION

In the present disclosure, the following terms may be understood with reference to the following:

The term “target region” may refer to a surgical site on which it is desired to operate;

The term “access direction” may refer to a direction in which a surgical tool approaches a target region;

The term “working direction” may refer to a direction in which a surgical tool operates on a target region, for example by applying a working load in the working direction. The working direction may be the angular deviation of the working tip (see below) from the access direction or from the longitudinal axis of the shaft;

The term “constrained joint space” may refer to a joint space in which the axes of the access direction and the working direction are not always collinear for every given target region;

The term “proximal end” may refer to the end of the instrument furthest from the target region, in use;

The term “distal end” may refer to the end of the instrument nearest to the target region, in use;

The term “working tip” may refer to a tool, end effector or other component at the distal end of the instrument, the working tip being used to operate on the target region. A non-exhaustive list of example working tips includes: perimeter cutters having a single blade, perimeter cutters having a circular blade, scrapers, burr removers, ablation tools including RF or acoustics (e.g. ultrasound), water jets, lasers, any combination of the above;

The term “operative portion” may refer to a distal portion of the instrument including at least one articulating portion and the working tip;

The term “lateral displacement of the working tip” may refer to the linear distance of the working tip from the access direction or from the longitudinal axis of the shaft, measured at a central location point at which the tool is mounted thereon and perpendicular to the longitudinal shaft axis;

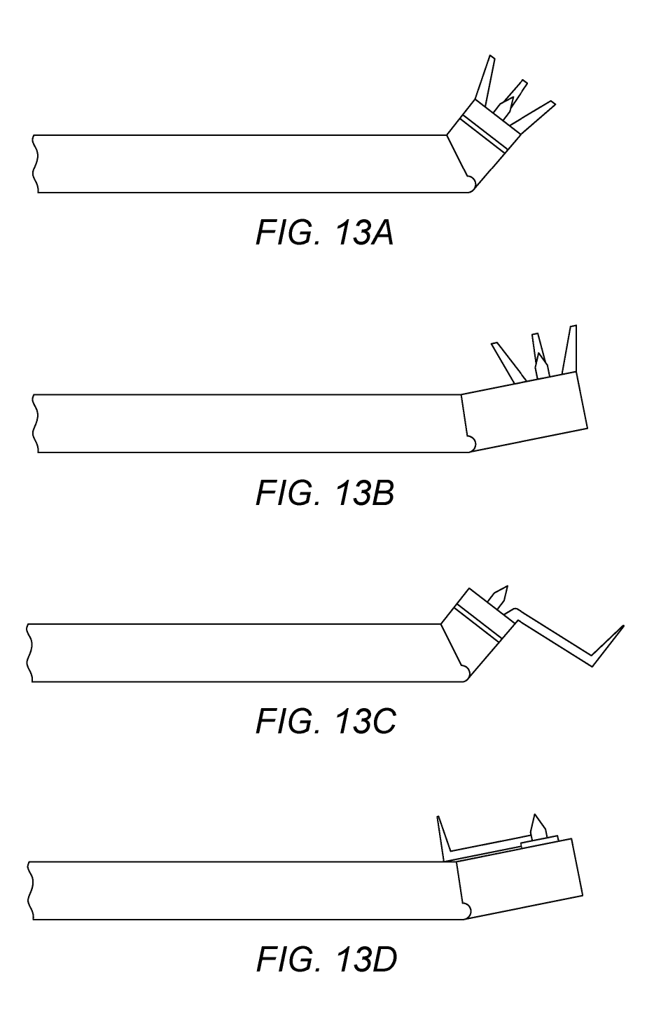

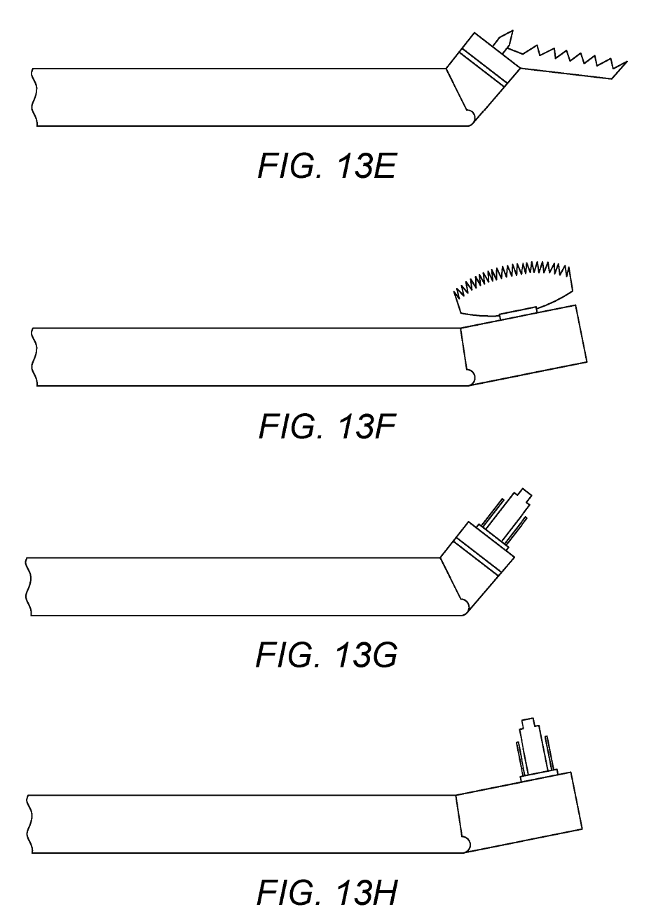

The term “central location point” may refer to the point at which the tool is mounted on the working tip. The central location point may be located on the distal end of the working tip (in the case of an end-mounted tool an example of which is in FIG. 13A) or on a side surface near the distal end of the working tip (in the case of a side-mounted tool an example of which is in FIG. 13B).

As described above, arthroscopic procedures for constrained joint spaces, such as the hip, are difficult using known techniques because of the difficulty in accessing the lesion to be treated.

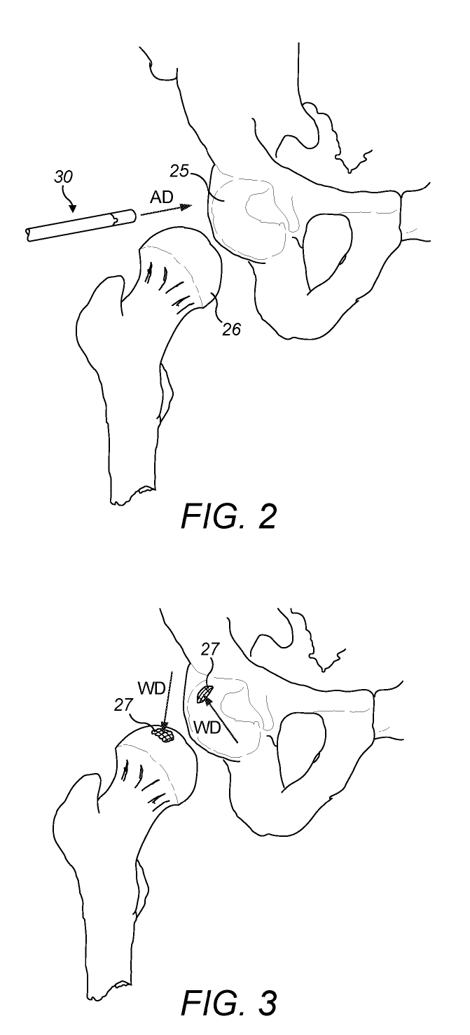

FIG. 2 shows a hip joint including an acetabulum 25 and femoral head 26. A surgical instrument 30 approaches the joint along an access direction AD. Lesions 27 to be treated are shown in FIG. 3. Each lesion 27 needs to be treated along a working direction WD. It can be seen that neither of the working directions WD indicated in FIG. 3 are collinear with the access direction of FIG. 2, there being no line of sight between the possible access directions and the required working directions. This is a problem addressed by embodiments of the claimed invention, as will be described in further detail below.

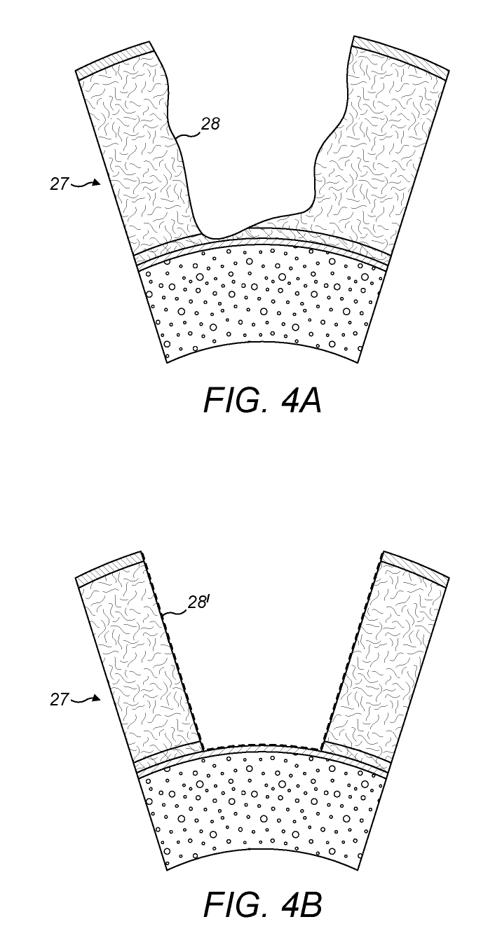

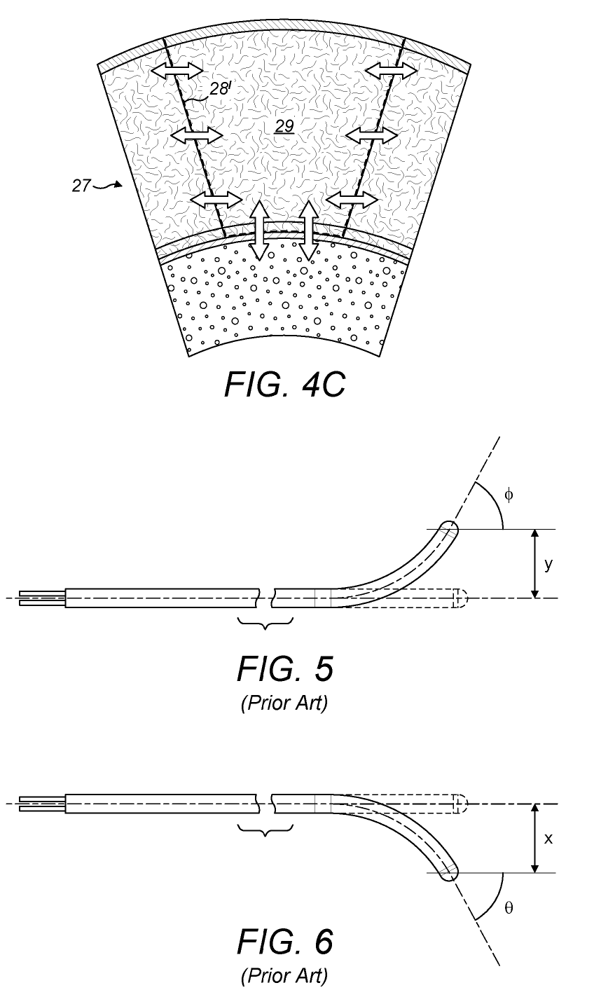

The aim of the arthroscopic procedure is illustrated in general terms in FIGS. 4A-4C. FIG. 4A shows a typical cartilage lesion whose edges 28 are frayed and uneven in terms of profile, mechanical and surface quality. The cartilage in the region of the lesion edge 28 will be degenerate and represents an unsuitable region around which to base a repair. This degenerate region may extend some distance (typically mm) away from the lesion edge 28, thus, if the repair is to be based around entirely undamaged tissue then the damaged region needs to be removed, despite the likelihood that it may appear healthy.

FIG. 4B shows the lesion 27 after the lesion edge or wall 28′ has been prepared. Degenerate cartilage has been removed to leave a prepared wall 28′ whose surface quality and geometry should optimise integration with a Regenerative Medicine Product (RMP). FIG. 4C shows the lesion 27 after delivery of the RMP 29.

The claimed invention can equally be used to treat lesions on the acetabulum, the femoral head or in any other constrained joint spaces such as the shoulder or ankle. In a constrained joint space, it is highly unlikely that the working direction will be collinear with the access direction. The claimed invention can also be used to treat lesions where there is a direct line of sight between access direction and target region such that the working direction is collinear with the access direction (for example in a knee joint).

FIGS. 5 and 6 (PRIOR ART) are reproduced from US2010280526. The working direction is the angular deviation of the working tip from the longitudinal axis of the shaft, this being selectable by the surgeon. Two different working directions are indicated in FIGS. 5 and 6, indicated as angles ϕ and θ respectively.

The lateral displacement of the working tip is the linear displacement of the working tip from the longitudinal axis of the shaft, this being dependent upon the selected working direction. The lateral displacement is indicated in FIGS. 5 and 6 by letters y and x respectively.

In the prior art, the working direction and lateral displacement are dependent upon one another. Therefore, for a working direction of θ, the only possible lateral displacement is x. Similarly, for a lateral displacement of y, the only possible working direction is ϕ.

The claimed invention improves the functionality of the instrument by permitting the working direction and lateral displacement to be selectable independently of one another, thus allowing the surgeon much greater freedom to select the optimum lateral displacement for any given working direction and vice versa.

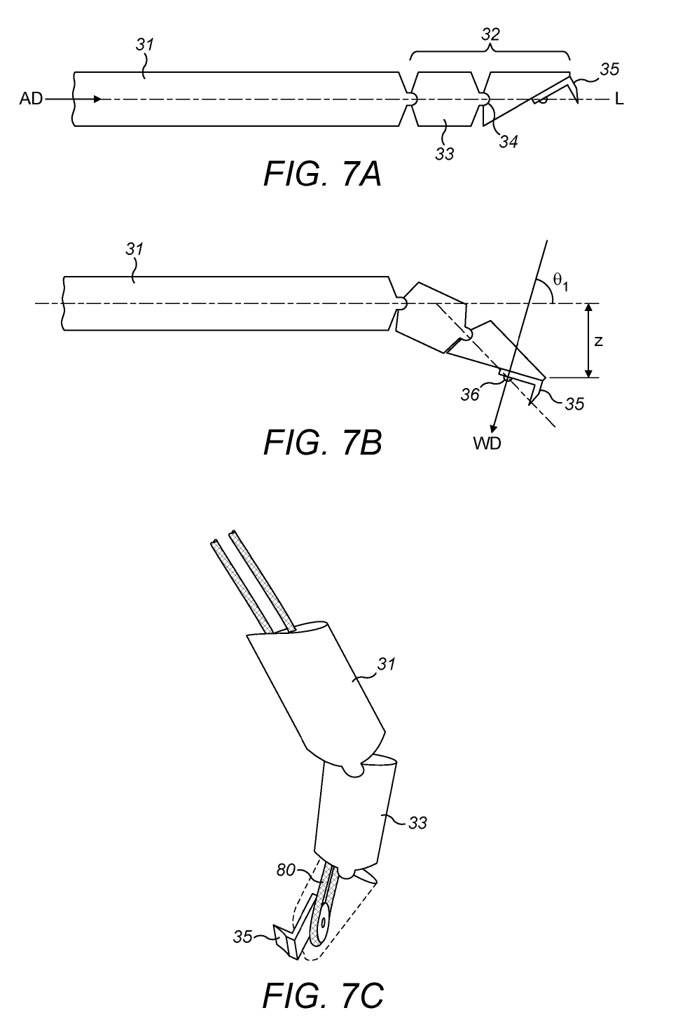

The surgical instrument 30 according to an embodiment of the invention comprises two assemblies: a handle assembly and a shaft assembly. FIGS. 7A and 7B show the distal end of the shaft assembly which includes a shaft 31 having a longitudinal shaft axis L and an operative portion 32. The shaft axis L is alignable with an access direction AD along which a target region of a joint (not illustrated) can be accessed, in use. In use, the shaft assembly is inserted into the handle assembly in a manner that will be described in more detail later.

At the distal end of the shaft is the operative portion 32 which, as shown in FIG. 87, is aligned with the longitudinal axis L of the instrument (and hence with the access direction AD).

The operative portion 32 includes at least one articulation segment 33 and a working tip 34. In some embodiments, the working tip and articulation segment are separate segments which articulate with respect to one another. However, it is possible for the articulation segment and the working tip to be fixed with respect to one another (e.g. as a unitary segment), so long as the operative portion as a whole can articulate with respect to the shaft. In the embodiment shown in FIG. 7A, the working tip includes a tool in the form of a cutting element 35 for preparing (by cutting) the target region. Other working tips and tools will be described below which have different functions, for example the delivery of RMP to the target region.

The operative portion 32 is selectively moveable between a first configuration shown in FIG. 7A and a second, deployed, configuration shown in FIG. 7B. Movement between the two configurations is by means of the articulation segment 33. More than one articulation segment may be present. The articulation segment 33 is caused to pivot with respect to the distal end of the shaft by means of, for example, a rod contained within the lumen of the shaft which is moveable axially by an actuator located at the proximal end of the shaft. In the aligned configuration, shown generally in FIG. 7A, it is preferable that none of the surgical instrument 30 is outside the maximum cross section of the shaft 31. That is, the operative portion 32, including the at least one articulation segment 33, the working tip 34 and the cutting element 35 (or indeed any other tool) do not extend beyond the widest point of the shaft 31.

In the deployed configuration shown in FIG. 7B, the operative portion 32 is no longer aligned with the longitudinal axis L of the instrument. A working direction WD has a direct line of sight to the target region and is the direction in which the tool acts upon the target region. The working direction is defined by the angle of articulation θ₁ with respect to the access direction and the longitudinal axis L which could, potentially, be zero (if both access and working directions are aligned). The lateral displacement z of the working tip is the linear displacement of the working tip 34 from the longitudinal axis L of the shaft. The working direction and the lateral displacement are independently selectable by the surgeon.

Locking means (not shown in FIG. 7B) may be used to lock the operative portion 32 in the deployed configuration with respect to the shaft 31 sufficiently firmly to handle a typical working load applied in the working direction when cutting at the target region. Alternatively, no locking means may be required, as is the case in one of the embodiments described in further detail below.

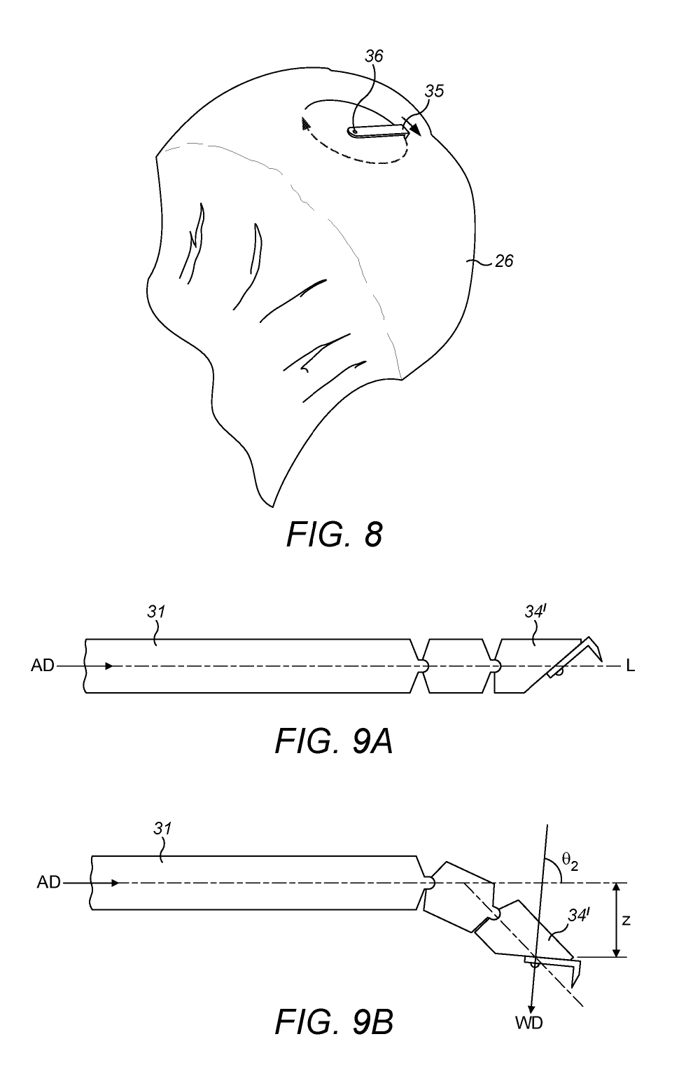

The cutting element 35 is moveable in a closed loop path centred on the central location point 36 so as to describe a circle around the location point 36. Example drive means 80 are indicated in FIG. 7C. As shown schematically in FIG. 8, the cutting element 35 is used to make a perimeter cut at the target region (in this case on the femoral head).

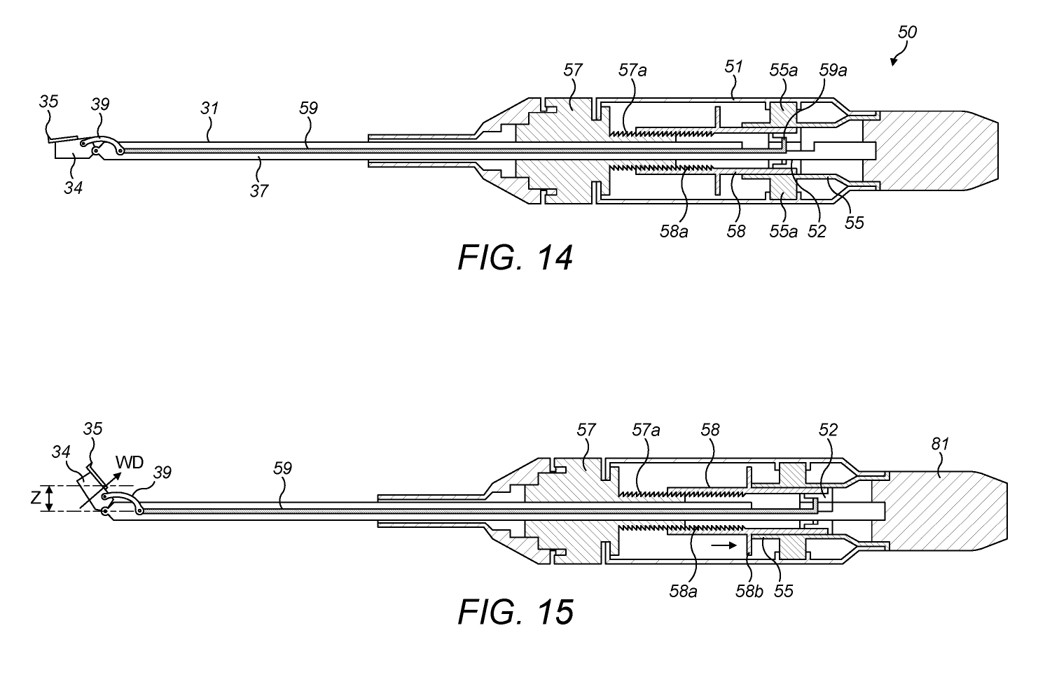

One way in which the working direction may be selectable independent of the lateral displacement is by removing the shaft assembly from the handle assembly and replacing with another shaft assembly that has a different working tip. This requires the presence of locator means for recording positional information relating to the shaft assembly so that said shaft assembly can be replaced in a repeatable position after removal from the handle assembly (for example using the embodiment illustrated in FIGS. 14-17). FIGS. 9A and 9B show a shaft 31 having a working tip 34′ which the cutting element 35 mounted on a differently-angled face (as compared with that of FIGS. 7A and 7B. This results in a different working direction, defined by angle θ2, for the same lateral displacement z.

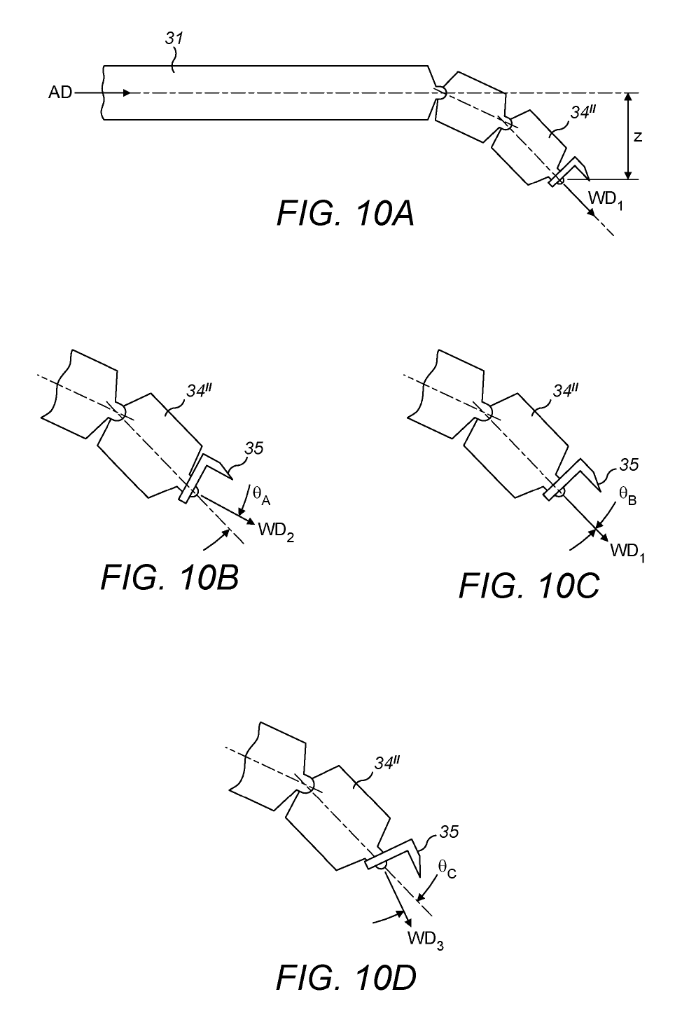

Another way in which the working direction may be selectable independent of the lateral displacement is shown in FIGS. 10A-10D in which, for a given lateral displacement z, the working direction can be varied by independently articulating the working tip 34″ and/or the cutting element 35 mounted thereon. In this way, a range of working directions WD₁, WD₂, WD₃ are available.

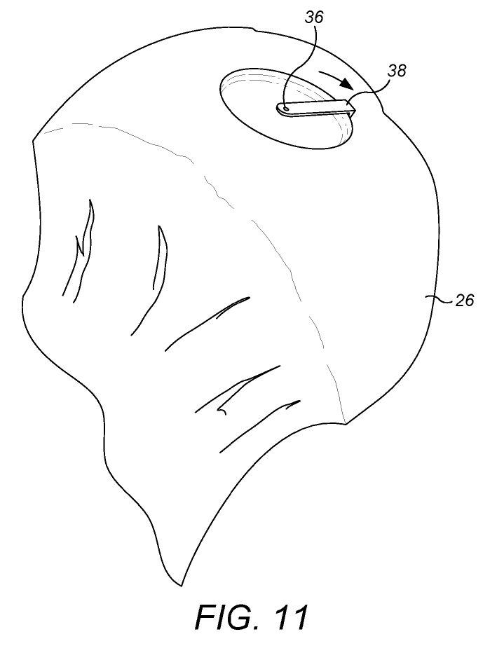

The cutting element 35 at the working tip is replaceable with other tools according to need. The diameter of the circle and hence the size of the perimeter cut is determined by the size of the cutting element 35 and is selected to cut well outside the lesion edges. A range of sizes of cutting element 35 would be available to the surgeon. The peripheral cutting element 35 can be replaced by an alternative type of cutting element 38 which has an abrading function shown in FIG. 11. This cutting element 38 is used to remove material from the area within the perimeter cut.

The cutting element described above is a blade, but other types of cutting element e.g. laser cutters or fluid jet cutters could equally be used. After the cutting is complete, the lesion wall has been prepared as previously shown in FIG. 4B.

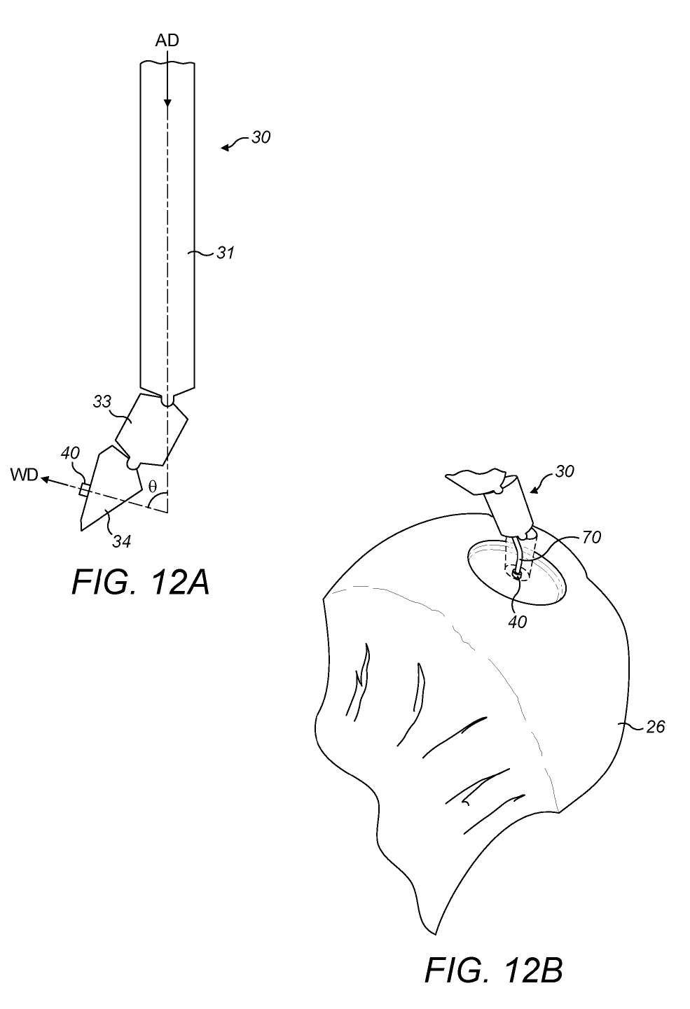

FIGS. 12A and 12B show a surgical instrument 30 in which the working tip comprises a nozzle 40 for the delivery of implantable material, such as RMP, to the target region. As before, the operative portion of the instrument is selectively moveable between a first configuration wherein the operative portion is aligned with the longitudinal shaft axis and a second, deployed, configuration (FIG. 12A). FIG. 12B shows the nozzle delivering RMP to a prepared target region on the femoral head 26.

The implantable material may be, but is not limited to being regenerative medicine product (RMP). The implantable material may be a powder, an injectable liquid, a morcellated solid, a gel, or any combination thereof, for example.

FIGS. 13A-13H show a selection of exemplary working tips that can be used in the claimed invention. These are:

FIG. 13A: a tripod for locating the centrepoint of the target region, mounted on the end of the working tip;

FIG. 13B: a tripod for locating the centrepoint of the target region, mounted on the side of the working tip;

FIG. 13C: cutting element for making a perimeter cut, mounted on the end of the working tip;

FIG. 13D: cutting element for making a perimeter cut, mounted on the side of the working tip;

FIG. 13E: cutting element for removing material from within the perimeter cut, mounted on the end of the working tip;

FIG. 13F: cutting element for removing material from within the perimeter cut, mounted on the side of the working tip;

FIG. 13G: delivery means for delivering implantable material to the target region, mounted on the end of the working tip; and

FIG. 13H: delivery means for delivering implantable material to the target region, mounted on the side of the working tip.

An embodiment of the instrument 30 will now be described in more detail in relation to FIGS. 14-17. FIGS. 14-17 show the instrument with its distal end at the left of the Figures and its proximal end at the right of the Figures.

FIG. 14 shows the instrument 30 in its first, non-deployed, position. The instrument comprises a housing assembly 50 and a shaft assembly including an elongate shaft 31 having a longitudinal axis and lumen 37 therethrough. An elongate actuation rod 59 is located in the lumen 37, the rod 59 serving as actuation means for the working tip 34. A pivotable distal rod link 39 connects the distal end of the rod 59 to the working tip 34. In this embodiment, there is only one articulating segment, this being the working tip 34 itself. A cutting element 35 is mounted on the working tip 34.

At or near the proximal end of the rod 59 is a rod link end 59a comprising a lug, boss, pin, or other formation extending radially from the rod 59. The rod link end 59a is captured within and is axially fixed with respect to a generally cylindrical rod link end housing 52. The rod link end housing 52 is located within a loading funnel 55 and is able to move axially with respect to the funnel 55. The funnel is provided with two orthogonally placed lugs 55a or other formations which engage with an interior surface of the housing 51 in order to lock the funnel 55 axially in the housing 51.

The rod link end housing 52 is moveable axially because of its engagement with an axially moveable locator tube 58. The engagement may be by virtue of a screw-thread (not illustrated), or other engagement means. The locator tube 58 is axially translatable along the longitudinal axis of the shaft. In the illustrated embodiment, the axial translation is possible because the locator tube 58 has an internal screw thread 58a at the distal end thereof. The internal screw thread 58a engages with an external screw thread 57a on the proximal end of a working tip position selector 57.

The working tip position selector 57 is rotatable about the longitudinal axis by the surgeon when the instrument is in use. Referring now to FIG. 15, rotation of the position selector 57, which is axially locked with respect to the housing 51, causes the locator tube 58 to travel axially in a proximal direction as indicated by the arrow in FIG. 15. The locator tube is rotationally locked with respect to the housing 51 by radial lugs or other formations 58b. These lugs 58b also serve as an axial endstop for the locator tube 58 as it reaches the distal end of the loading funnel 55, as shown in FIG. 15.

As the locator rod 58 moves axially, it brings with it the rod link end housing 52 and therefore the rod link end 59a and rod 59. Axial movement of the rod 59 in the proximal direction pulls the distal rod link 39 in order to effect articulation of the working tip 34 to a deployed position shown in FIG. 15 having a desired lateral displacement z and working direction WD.

Rotation of the position selector 57 in the opposite direction causes the locator tube 58 to travel in a distal direction, moving the rod 59 in a distal direction back towards the position shown in FIG. 14. Further rotation of the position selector 57 causes the rod link 39 to pivot downwardly (not illustrated) offering further range of movement of the working tip to select a desired lateral displacement. The position selector 57 itself may serve as an endstop for axial movement of the locator tube 58 in the distal direction, when it reaches the distal end of screw thread 57a.

When the desired lateral displacement and working direction are obtained, the cutting element 35 can be actuated by the surgeon operating a working tip actuator dial 81 at the proximal end of the instrument. The actuator dial 81 operates drive means 80 (not shown) which are in communication with the cutting element via the lumen 37 of the shaft 31. Additional locking means (not shown) could be provided to hold the selected lateral displacement and working direction against the working load, although additional locking means are generally not necessary as the pitch of the screw threads 57a, 58a is narrow enough to prevent undesired relative movement thereof.

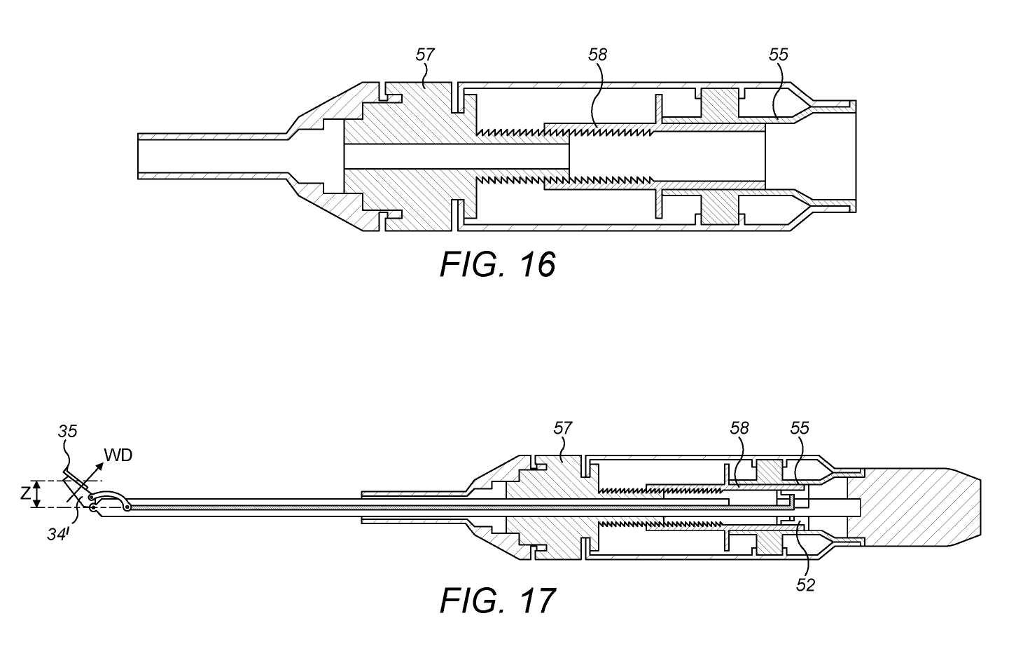

When it is desired to provide a different working tip, in order to provide a different tool or a different working direction, the working tip actuator dial 81 can be removed so that the entire shaft assembly can be removed via the proximal end of the handle assembly 50. This condition is illustrated in FIG. 16. Whilst the shaft assembly is removed, the relative axial positions of the position selector 57, locator tube 58 and loading funnel 55 are retained and unchanged. Therefore, when the same or another shaft assembly is loaded into the handle assembly, as in FIG. 17, the rod link end housing 52 thereof abuts the unmoved locator tube 58, allowing the previous positioning of the shaft assembly to be restored. FIG. 17 shows the condition in which the working direction WD is different compared with FIG. 15 because of the differently-angled working tip 34′ that has been selected. The lateral displacement z′ may also be different compared with FIG. 15.

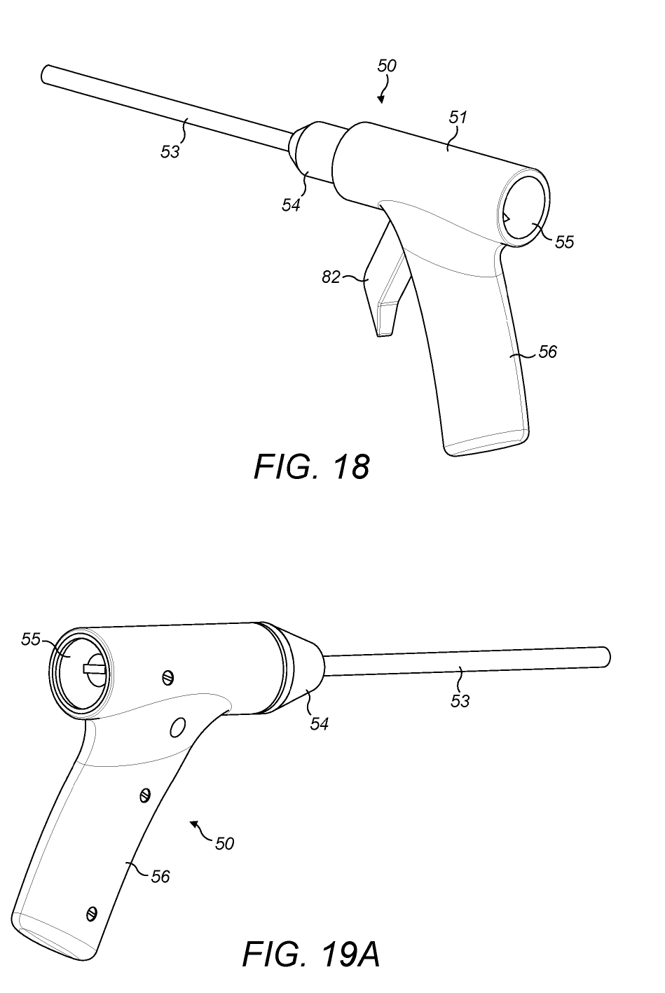

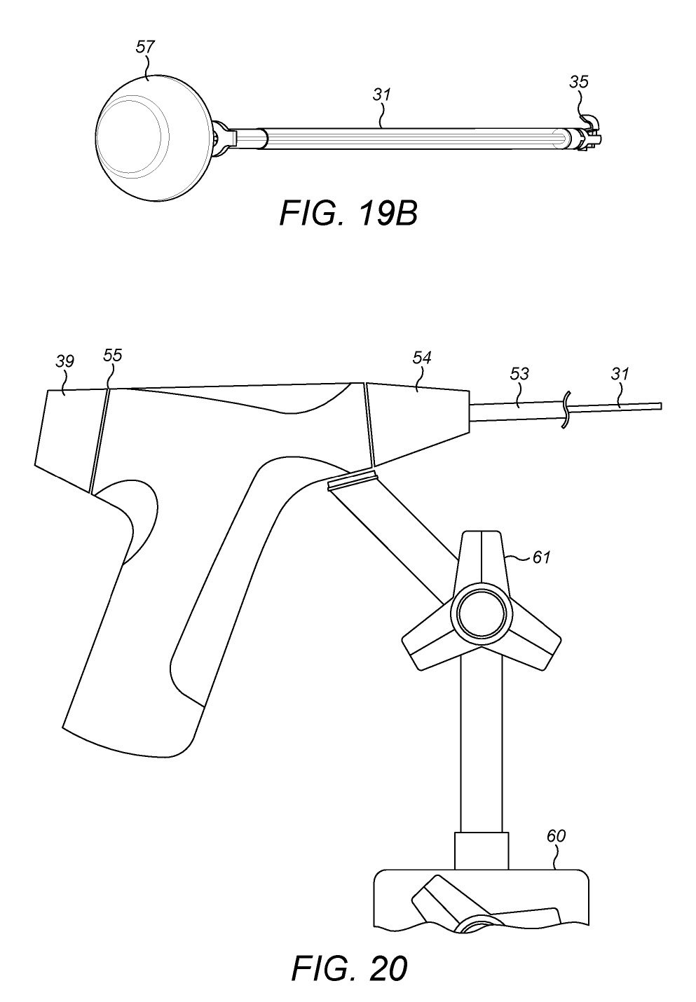

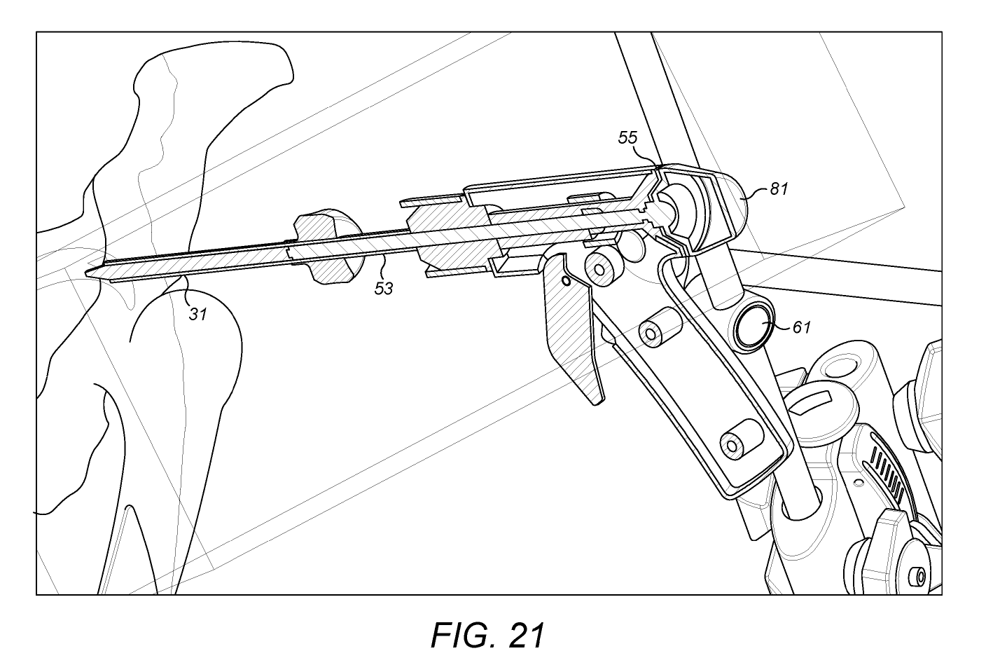

Alternative embodiments of the housing are shown in FIGS. 18-22. In FIGS. 18 and 19, the handle assembly 50 comprises a housing 51 having a pistol-type grip 56 and a trigger-type actuator 82. An axially-extending shaft locator 53 is a cylindrical tube of greater diameter than the shaft 31 and through which the shaft can be inserted via a loading funnel 55 located at the rear of the housing 51. A portal bridge 54 is provided which, in use, mates with an arthroscopy portal. This ensures that the instrument 30 is firmly seated against the portal and provides further stability during operation. The housing assembly can be clamped to an operating table clamp 60 or the like.

The shaft 31 and its associated operative portion 32 (in the first, undeployed, configuration) is insertable into the housing assembly via loading funnel 55. A drive dial (working tip position selector) 57 locates against the proximal end of the housing when the shaft assembly is fully inserted. Typically the first-selected operative portion is a tripod of the type shown in FIG. 13A or 13B which can be used to pinpoint the centre of the target region.

The shaft assembly advances towards the target region along an access direction. As the operative portion approaches the target region, the surgeon actuates the instrument to move the operative portion into the deployed position so that the operative portion is now aligned with a suitable working direction for the target region concerned. As shown in FIG. 20, the surgeon can adjust the position of the whole surgical instrument by moving the adjustable arm clamp 61 and/or table clamp 60 until the desired position of the operative portion at the target region (which may not have a direct line of sight with the access direction) is reached. Then the clamps are tightened so that the position of the handle assembly is fixed with respect to the operating table and the patient.

Once the handle assembly position is fixed, the whole shaft assembly including the operative portion can be removed from the target region by withdrawing it through the handle assembly, exiting through the loading funnel 55.

The first shaft assembly can then be switched for a second shaft assembly having a cutting element at its working tip. Other than the different working tip, the dimensions of the second shaft assembly are the same as those of the first shaft assembly, in particular the length of the shaft assembly along its longitudinal axis is the same. Therefore, when the second shaft assembly is inserted into the housing assembly, the operative portion of the second shaft assembly will arrive at the same, defined, position at the target region that is desired. The interaction between the drive dial 57 and the loading funnel 55 and the interaction between the shaft locator 53 and the shaft 31 serve as locator means for recording positional information relating to the shaft assembly so that it can be repeatably removed and replaced in the housing assembly.

The second shaft assembly includes cutting element of the type shown in FIG. 13C or 13F which can make a perimeter cut at the target region.

Next, with the handle assembly still clamped in place, the second shaft assembly can be removed and replaced with a third shaft assembly, this time having a cutting element of the type shown in FIG. 13E or FIG. 13F which can remove material from an area within the perimeter cut. The target region is now prepared to receive implantable material such as RMP.

Next, with the handle assembly still clamped in place, the third shaft assembly can be removed and replaced with a fourth shaft assembly, this time having delivery means of the type shown in FIG. 13G or 13H. This shaft assembly can deliver RMP to the target region. Still further shaft assemblies may be used after, for example having a wiper tool which can wipe excess RMP from the target region and/or to compress the RMP within the target region. A separate compressor tool may be used before or after the wiper tool to compress the RMP within the target region. A curing tool which can cure implantable material delivered to the target region may be used after delivery of the RMP to the target region. In every case, the positioning of the operative portion of the shaft assembly is accurately repeatable in view of the positional information recorded by the locator means components.

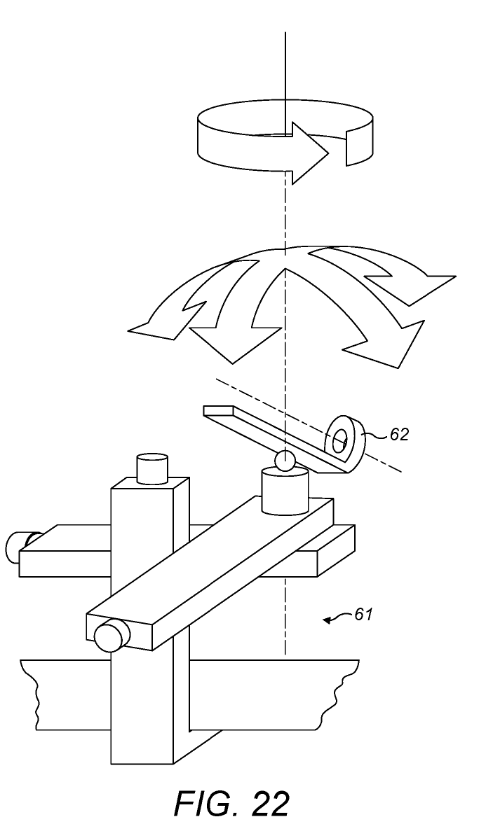

In a further embodiment illustrated in FIG. 22, the positional information could be recorded by an adjustable instrument support 62 attached to an adjustable arm clamp 61. The position of the instrument support 62 can be set up by adjusting in any of the directions indicated by arrows in FIG. 22 and then locked in place with respect to the arm clamp 61. The handle assembly can optionally be clamped to the instrument support 62. Then the shaft assembly and/or entire surgical instrument 30 (i.e. handle assembly and shaft assembly) can be removed and repeatably replaced.

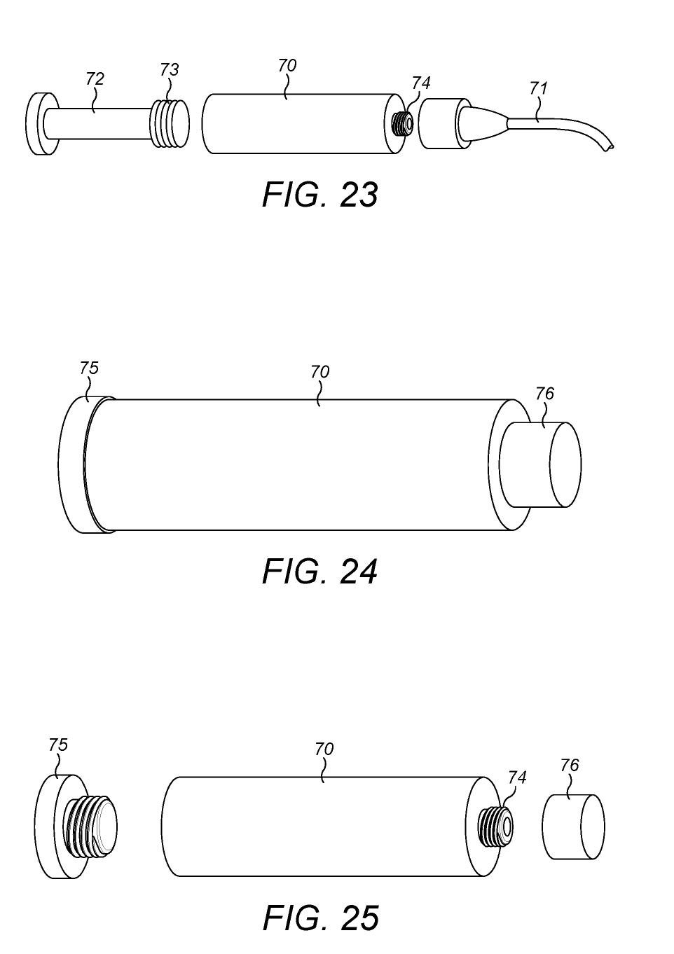

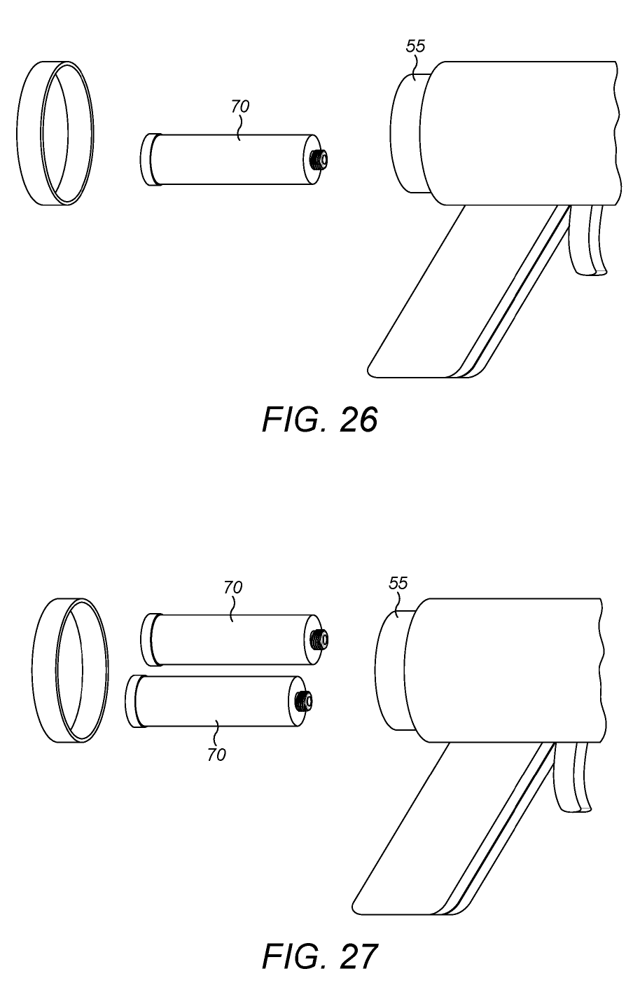

Exemplary details of the delivery of RMP to the target region will now be described with reference to FIGS. 12A-12B and 23-27.

The nozzle 40 of the delivery means is connected to a reservoir 70 containing implantable material, e.g. RMP by means of a tube 71 which passes through the lumen of the shaft 31. The reservoir 70 is typically located within the handle assembly but could possibly be located within the lumen of the shaft 31 itself or located near the distal end of the shaft.

The reservoir 70 is similar to the barrel of a typical syringe and includes a plunger 72 which, when moved axially into the chamber of the reservoir, causes a piston 73 to expel RMP from the reservoir through an outlet 74. The chamber may have more than one compartment, separated by a frangible membrane so that different components of medicament can be stored separately and then mixed shortly before, or upon delivery.

The reservoir 70 may form part of a medicament cartridge (illustrated in FIGS. 24 and 5) which is supplied as shown in FIG. 24, having end caps 75, 76 to seal the RMP within the reservoir. The cartridge may be single-use or refillable and one or more cartridges can be loaded into the handle assembly via the loading funnel 55 as shown in FIGS. 26 and 27.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### REFERENCE NUMERALS

### Prior Art

2 arthroscope
4 skin
6 ligaments
8 patella
100 knee
10 target region
12 femoral condyles
14 femur
16 axis of access direction
18 tibia
20 fibula
22 tibial plateau
ϕ working direction in prior art
Θ working direction in prior art

### Distal End/Working Tip Structure

25 acetabulum
26 femoral head
27 lesions
28 edges of lesion
28′ prepared lesion edge or wall
29 RMP
30 surgical instrument
31 shaft
L longitudinal shaft axis
32 operative portion
33 articulation segment
34, 34′, 34″ working tips
35 cutting element
36 central location point
37 lumen
38 alternative abrading cutting element
40 nozzle
AD access direction
WD working direction

### Proximal End/Working Tip Positioning

50 handle assembly
51 housing
52 rod link end housing
53 shaft locator—front housing/cannula
54 portal bridge—front housing/cannula
55 loading funnel
55a loading funnel lugs
56 pistol grip
57 working tip position selector
57a position selector screw thread
58 locator tube
58a locator tube distal screw thread
58b locator tube lugs
59 actuation rod
59a proximal rod link end
39 distal rod link
60 operating table clamp
61 adjustable arm clamp
62 instrument support

### Working Tip Actuation

80 drive means
81 drive dial/working tip actuator
82 trigger-type actuator

### Delivery of RMP

70 reservoir
72 plunger
73 piston
74 outlet
75,76 end caps

1. A surgical instrument for minimally-invasive arthroscopic use in a constrained joint space, the surgical instrument comprising:
a handle assembly;
a shaft assembly including:
a shaft having a longitudinal shaft axis and a lumen therethrough and being connected to the handle assembly at a proximal end of the shaft; and
an operative portion including a working tip with a tool mounted thereon at a distal end of the shaft,
wherein, in use, the operative portion is selectively moveable between a first configuration in which the operative portion is substantially aligned with the longitudinal shaft axis, and a second, deployed, configuration in which at least part of the operative portion has:
a selectable working direction defined by an angle between the tool and the longitudinal shaft axis; and
a selectable lateral displacement defined by a linear distance between the working tip and the longitudinal shaft axis, measured at a central location point at which the tool is mounted thereon and perpendicular to the longitudinal shaft axis,
the working direction and lateral displacement being selectable independently of one another.

2. The surgical instrument of claim 1, wherein the operative portion includes an articulation segment which is said working tip or is intermediate said working tip and said shaft, the articulation segment having actuation means capable of moving the operative portion between the first configuration and the deployed configuration.

3. The surgical instrument of claim 2, wherein the actuation means comprises one or more rods, cables, pneumatic or hydraulic means located within the shaft lumen and actuatable via said handle.

4. The surgical instrument of claim 3, wherein the actuation means comprises an actuation rod.

5. The surgical instrument of claim 4, wherein the actuation means includes a rod link at a distal end of and pivotable with respect to said actuation rod.

6. The surgical instrument of claim 5, wherein said actuation rod has a radially extending formation or rod link end at or near a proximal end thereof, the formation being axially fixed with respect to a rod link end housing and axially moveable therewith so as to operate said articulating element.

7. The surgical instrument of claim 6, further comprising a locator tube which is selectively axially translatable along the longitudinal shaft axis, the locator tube being capable of moving the rod link end housing axially.

8. The surgical instrument of claim 7, wherein the locator tube is selectively axially translatable along the longitudinal shaft axis by virtue of a screw thread at one end thereof.

9. The surgical instrument of claim 8, wherein the locator tube screw thread engages a screw thread on a rotatable position selector, wherein rotation of the rotatable position selector causes the axial translation of the locator tube.

10. The surgical instrument of claim 1, further comprising locking means which, in said deployed configuration, lock the operative portion in the deployed configuration with respect to the shaft in order that a working load can be applied in the working direction.

11. The surgical instrument of claim 10, wherein said working load is in the range 3N to 10N.

12. The surgical instrument of claim 2, wherein the lateral displacement is selectable by selecting an angle of articulation of the articulation segment with respect to the longitudinal shaft axis.

13. The surgical instrument of claim 1, wherein the working direction is selectable by selecting the angle at which the tool is mounted on the working tip.

14. The surgical instrument of claim 1, wherein the working direction is selectable by adjusting the angle at which the tool is mounted on the working tip.

15. The surgical instrument of claim 1, wherein the shaft assembly is removable and replaceable via the proximal end of the handle assembly.

16. The surgical instrument of claim 15, further comprising locator means for recording positional information relating to the shaft assembly so that said shaft assembly or another shaft assembly can be replaced in a repeatable position after removal from the handle assembly.

17. The surgical instrument of claim 16, wherein the locator means comprises a locator tube whose axial position remains unchanged while the shaft assembly is being removed and replaced.

18. The surgical instrument of claim 1, wherein the constrained joint space is a hip joint space.

19. The surgical instrument of claim 1, wherein the tool is moveable in a closed loop path with respect to the central location point fixed with respect to the shaft.

20. The surgical instrument of claim 19, wherein said central location point is on an axis aligned with said working direction.

21. The surgical instrument of claim 1, wherein said tool comprises a cutting element capable of producing a cut surface at said target region, the cut surface having one or more selectable characteristics including one or more of size, depth, orientation, shape and surface topography.

22. The surgical instrument of claim 21, wherein the cutting element is a blade, a laser cutter, ultrasonic cutter or a fluid jet cutter.

23. The surgical instrument of claim 21, wherein the cutting element is moveable in a closed loop path with respect to a central location point fixed with respect to the shaft, so as to make a perimeter cut along said closed loop path at said target region.

24. The surgical instrument of claim 23, wherein the cutting element is capable of cutting to remove material from an area within said perimeter cut.

25-51. (canceled)
